(19) United States
(12) Patent Application Publication
(10) Pub. No.: US 2025/0127639 A1
(43) Pub. Date: Apr. 24, 2025

(54) WOVEN BRAID PATTERN FOR STENTS AND RELATED METHODS

(71) Applicant: Merit Medical Systems, Inc., South Jordan, UT (US)

(72) Inventors: Zeke Eller, Plano, TX (US); Nick Poulides, South Jordan, UT (US)

(21) Appl. No.: 18/918,984

(22) Filed: Oct. 17, 2024

Related U.S. Application Data
(60) Provisional application No. 63/591,948, filed on Oct. 20, 2023.

(51) Int. Cl. A61F 2/90; A61F 2/95

(52) U.S. Cl. CPC A61F 2/90; A61F 2/9517; A61F 2002/9528; A61F 2230/0091; A61F 2250/0039

(57) ABSTRACT

Stents described herein can comprise a tubular body having a midbody that extends to a first end and also extends to an opposing second end. The midbody can include a helical thread arranged along at least a portion of its length. The helical thread includes a plurality of turns with an interthread space disposed between the turns. The stent is formed from a plurality of wires woven or braided to form a mesh structure and a twisted wire structure. The twisted wire structure includes one or more pairs of wires longitudinally twisted together. The twisted wire structure can be disposed at one or more of the interthread space, the first end, and the second end.

### CROSS-REFERENCE TO RELATED APPLICATIONS

This application claims priority to U.S. Provisional Patent Application No. 63/591,948, entitled “WOVEN BRAID PATTERN FOR STENTS AND RELATED METHODS,” filed on Oct. 20, 2023, which is incorporated herein by reference in its entirety.

### TECHNICAL FIELD

This application generally relates to medical devices. More particularly, this application relates to stents having woven braid patterns forming particular structural features.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of a stent in accordance with an embodiment.

FIG. 1A is an expanded side view of a portion of the stent of FIG. 1 indicated in FIG. 1 as 1A.

FIG. 2A is a side view of a woven braid of the stent of FIG. 1.

FIG. 2B is a side view of another woven braid of the stent of FIG. 1.

FIG. 2C is a side view of another woven braid of the stent of FIG. 1.

FIG. 2D is a side view of another woven braid of the stent of FIG. 1.

FIG. 2E is a side view of another woven braid of the stent of FIG. 1 having a flange disposed at an end.

FIG. 3 is a side view of the stent of FIG. 1 with a cover disposed over the stent.

FIG. 4 is side view of a portion of another embodiment of a stent.

### DETAILED DESCRIPTION

Stents are disclosed herein. In some embodiments, the stents described herein comprise a tubular body having an interior dimension and comprising a midbody that extends to a first end and also extends to an opposing second end. In certain embodiments, the midbody includes a thread arranged helically along at least a portion of its length. The thread includes a plurality of turns with an interthread space disposed between the turns. The stent is formed from a plurality of wires. The wires form a mesh structure and a twisted wire structure. The twisted wire structure can be disposed at one or more of the interthread space, the first end, and the second end. The positioning of the twisted wire structure is selected to enhance particular aspects of performance when the stent is disposed within a delivery catheter. For example, the twisted wire structure can provide an elongation of less than 150%, such as when the stent is constrained within a 6.5 French diameter to 8.5 French catheter. Though stents may be described herein with reference to placement in the bile duct, this should be understood as one exemplary use. Stents in accordance with the present disclosure can be used in a number of placements, including but not limited to, gastrointestinal, colonic, esophageal, pulmonary, vascular, pancreatic, and ureteral placements.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As applied to a stent deployed within a bile duct, the proximal end of the stent refers to the end closest to the patient's liver, and the distal end of the stent refers to the opposite end, the end closer to the patient's duodenum.

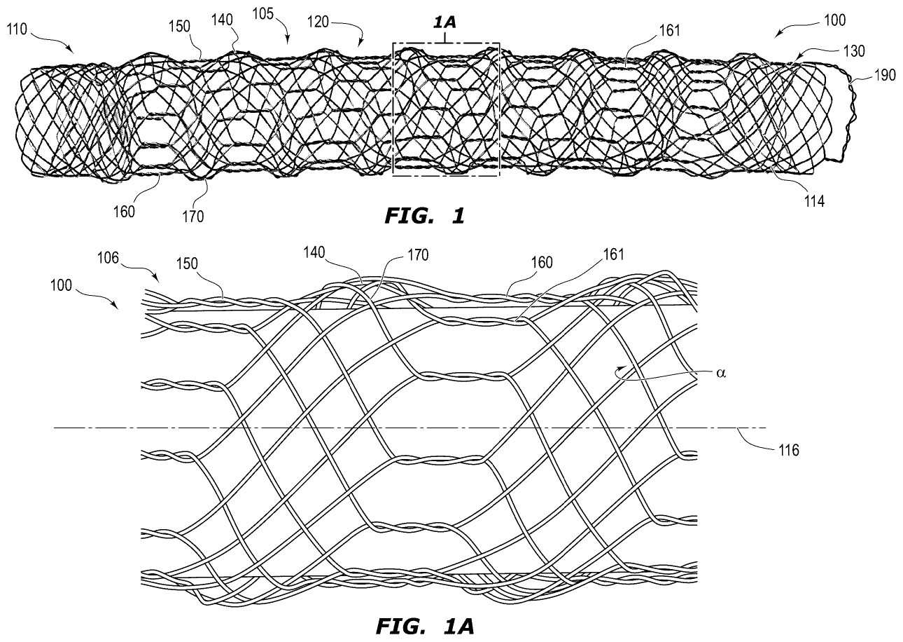

FIG. 1 and FIG. 1A depict a stent 100 having a generally tubular stent body 105 that comprises a midbody or a midbody portion 120 extending to two opposing ends or end portions, i.e., a first end 110 and a second end 130, which combine to define a lumen or an interior space passing through the stent 100. The first end 110 and the second end 130 can respectively be considered the proximal end and the distal end of the stent for purposes of the descriptions herein, unless expressly stated otherwise. The stent 100 includes a helical thread 140 arranged circumferentially along at least a portion of the outer surface of at least the midbody 120. In some embodiments as illustrated in FIG. 1 and FIG. 1A, the midbody 120 comprises a helical thread 140 and an interthread space 150, which together define the portion of the interior space between the ends 110, 130 of the stent 100.

In some embodiments, the overall length of the stent 100 may range from about 20 millimeters to about 250 millimeters, including ranging from about 20 millimeters to about 90 millimeters, or from about 60 millimeters to about 160 millimeters. In particular examples, the stent 100 can have an overall length of about 100 millimeters and the midbody can have a length of about 50 millimeters, an overall length of about 120 millimeters and a midbody length of about 70 millimeters, or an overall length of about 150 millimeters and a midbody length of about 100 millimeters. The overall diameter of the stent 100 can be from about six millimeters to about 45 millimeters. In particular embodiments, the overall diameter is about four millimeters to about 32 millimeters, or from about six millimeters to about 12 millimeters, or from about eight millimeters to about ten millimeters.

As illustrated in FIG. 1 and FIG. 1A, the helical thread 110 can be formed as an outward expansion of the structure of the stent 100, such that the helical thread 140 protrudes radially from the portion of the stent 100 on which it is located (e.g., the midbody 120). Therefore, in an aspect of the present disclosure, the helical thread 140 can constitute a primary contact surface between the stent 100 and the surrounding tissue of a body lumen in which it is placed, whereby the helical thread 140 allows the stent 100 to grip the tissue more effectively. This grip aids in anchoring the stent 100, therefore the helical thread 140 can enhance the stent's 100 resistance to migration within the body lumen, e.g., bile duct. While not bound by any particular theory, due to its helical arrangement, the helical thread 140 can function somewhat as a screw thread in the classical sense, i.e., providing conversion between longitudinal motion and rotational motion. More specifically, forces acting on the stent 100 that would tend to produce longitudinal migration are instead translated into rotational forces, thereby greatly reducing longitudinal displacement of the stent 100.

In another aspect, the inclusion of the helical thread 140 may also aid in fluid flow through stented regions. For example, spiral laminar flow is a predominant type of arterial flow and is commonly seen in narrowing passages in the circulatory system. While not bound by any particular theory, the helical arrangement of the helical thread 140 can facilitate or enhance such flow in certain anatomical structures.

In other aspects, the helical thread 140 can enhance the mechanical properties of the stent 100. In one aspect, the helical thread 140 increases the axial and radial strength of the stent 100. This can be realized to a greater degree as the stent length increases, as compared to conventional unthreaded stents in which radial and axial strength decrease more drastically with increasing length. In another aspect, the helical thread 140 can decrease the stent's 100 resistance to lateral flexion, i.e., its bending force. Stated differently, the turns of the helical thread 140 provide flexion points that allow the stent 100 to bend more easily. The helical thread 140 also enables the stent body 105 to remain more open when placed in narrowing and tortuous anatomy, resisting collapsing/infolding. This can make the stent 100 more suited for navigating small and/or tortuous anatomy while keeping the lumen open and exerting less pressure on said anatomy.

In various embodiments, the stent 100 may comprise one or more wires 114 formed from any suitable material known in the art, including metals, alloys thereof, and polymers. In some embodiments, the material may be a shape memory alloy, including but not limited to an alloy of nickel and titanium commonly known as Nitinol. In one example, the stent 100 is made using “DFT wire” (drawn filled tubing) which includes a Nitinol outer sheath and a core containing platinum to give fluoroscopic visibility or radiopacity to provide a fluoroscopic or radiopaque marker. Other metals include magnesium, zinc, and iron. In certain embodiments, the stent 100 can comprise other types of fluoroscopic markers including gold foil or a platinum-iridium band swaged onto the wire 114. Other radiopaque markers are within the scope of this disclosure. In some embodiments, the stent 100 can comprise a biodegradable material.

In certain embodiments, the diameter of the wire 114 may be from about 102 micrometers to about 381 micrometers, or more particularly from 140 micrometers to about 254 micrometers. In other embodiments, the diameter may be from about 165 micrometers to about 178 micrometers. Generally speaking, smaller wires may be used with smaller diameter stents and larger diameter wires may be used with larger diameter stents. Also, while smaller diameter wires can be used for stents designed for “through the scope” placement, stents designed for “over the wire” placement can include wires having significantly larger diameters.

As illustrated in FIG. 1 and FIG. 1A, the stent 100 includes a woven braid 106 having a twisted wire structure 160 and a grid or mesh structure 170. The twisted wire structure 160 includes at least one twisted wire pair 161 where two of the wires 114 are longitudinally twisted together to form a double helix. In other embodiments, the twisted wire structure 160 may include three, four, five or more of the wires 114 longitudinally twisted together. In some embodiments, the twisted wire structure 160 can include a plurality of twisted wire pairs 161. For example, the twisted wire structure 160 may comprise from four to 20, from eight to 16, and from ten to 12 twisted wire pairs 161 equidistantly distributed about the circumference of the stent body 105. In certain embodiments, the number of twisted wire pairs 161 can change along the length of the stent body 105 to improve functional/clinical performance. For example, at the cystic duct entry to the bile duct, the number of twisted wire pairs 161 may change from 12 twisted wire pairs 161 down to 6 or 4 so that one or more drainage holes can be cut in a cover of the stent 100 to better promote drainage from the cystic duct into the bile duct without the stent 100 blocking the drainage flow.

As illustrated in FIG. 1 and FIG. 1A, the mesh structure 170 can be formed of the wires 114 braided or woven according to a braid pattern. More specifically, the wires 114 can be arranged in a particular braid pattern having a pitch, and with a braid angle a that can be constant over a given region of the stent 100 and also vary over other regions to provide certain shape and strength characteristics. The pattern comprises repeating structural units, the most basic of which being cells, which are the openings formed by sets of adjacent crossing points of the wires 114.

In some embodiments, the braid pattern of the mesh structure 170 is a one-wire, two-over, two-under braid pattern (referred to as a “one over two” pattern), which means that a single strand passes over two strands (or two different portions of itself, such as in a single wire braid design) and then under two other strands (or yet two other portions of itself, such as in a single wire braid design). Alternative braid patterns may be used as well, such as a one-wire, one-over, one-under braid pattern (referred to as a “one over one” pattern). Other possible braid patterns include the diamond two-wire, one-over, one-under braid pattern and the diamond two-over, two-under braid pattern. Two strands can also be ‘hooked,’ or linked, together at their crossing point rather than passing over/under one another uninterrupted.

As shown in FIG. 1A, the braid angle a is an angle formed by a given wire 114 of the mesh structure relative to the longitudinal axis 116 of the stent 100. A larger (higher) braid angle, approaching, for example, 90 degrees, results in a higher pic count (number of points of intersection of the strands) per given longitudinal length (e.g., an inch) of a given braid (or weave) pattern. These parameters can be varied to impart certain characteristics to the stent body 105. A higher pic count can produce greater stiffness (i.e., a lower degree of compressibility). A smaller (lower) braid angle results in a lower pic count per given longitudinal length, which can result in greater softness (i.e., less stiffness and a higher degree of compressibility). In some embodiments, the braid angle a is from about 35 degrees to about 90 degrees. In certain of such embodiments, the braid angle a in the end portions 110, 130 is from about 40 degrees to about 60 degrees, and the braid angle a on the helical thread 140 is from about 40 degrees to about 80 degrees. In an aspect, the braid angle a on the helical thread 140 can vary based on thread height/geometry and starting angle from the midbody 120.

The pitch (i.e., axial distance between intersecting strands) also impacts the compressibility and stiffness of the mesh structure 170. In an aspect, a decrease in pitch, i.e. tighter pitch, may correlate with an increase in migration resistance of the stent 100. The pitch is related to the number of wires 114 woven or braided together and the braid angle a, and therefore can vary over different geometries.

The wires 114 of the mesh structure 170 may be braided or woven in a given pattern in accordance with an appropriate braid design, such as a closed-loop braid design, a single wire woven design, an endless braid design, or the like. In some embodiments, the wires 114 are braided in a closed-loop braid design in which multiple strands are interlaced in a first direction (e.g., a distal direction) and then turn and are interlaced back in an opposite second direction (e.g., back in the proximal direction). In still other embodiments, the mesh structure may have an endless braid design in which multiple strands are interlaced. In some embodiments, the braid pattern can comprise hook stitches. In other embodiments, a “hook and cross” braid pattern is used in which the pattern includes both hook stitches and cross stitches. In some embodiments, the braid pattern is created using an axial braiding approach. In some embodiments, the braid pattern is created using a radial braiding approach.

The wires 114 may include varying numbers of wires, where the number used can depend in part upon the size of the stent 100 and the braid or weave pattern. In some embodiments, the stent 100 includes a wire count from 12 to 72 wires, or more particularly from 16 to 32 wires, or more particularly from 16 to 24 wires. Wire counts increase by a factor of 4 with 1-over-1 braid patterns and by a factor of 8 with 1-over-2 braid patterns.

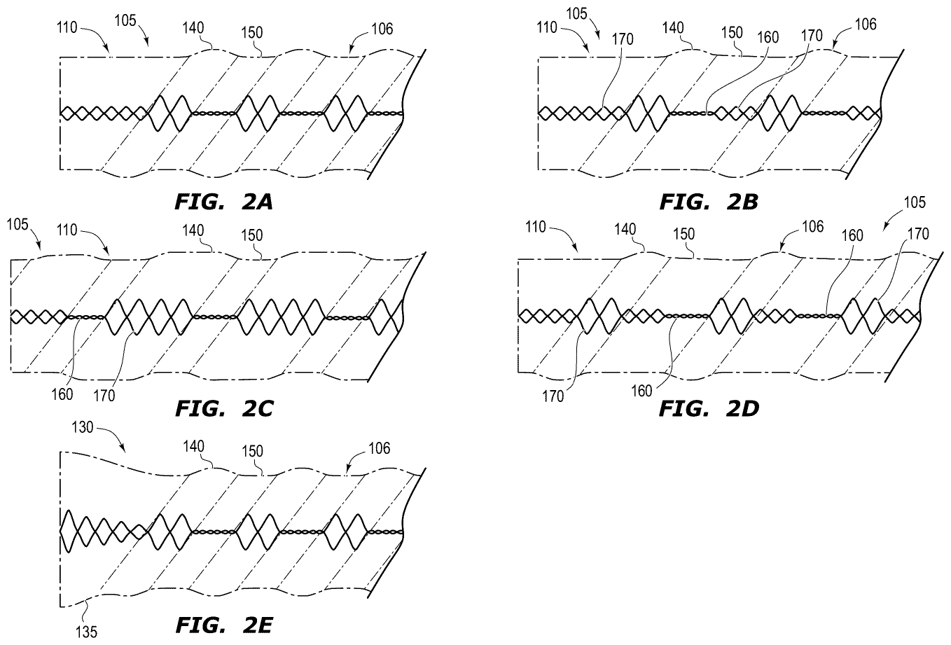

FIGS. 2A-2D illustrate various patterns of the woven braid 106 including the twisted wire structure 160 and the mesh structure 170 distributed along the length of the stent body 105. As illustrated in FIG. 1, FIG. 1A, and FIG. 2A, the twisted wire structure 160 is disposed in the interthread space 150 and the mesh structure 170 is disposed at the helical thread 140 and at the ends 110, 130. In other embodiments, the twisted wire structure 160 can be disposed relative to the mesh structure 170 in any suitable configuration to provide desired physical characteristics of the stent 100, such as elongation, flexibility, compressibility, etc. For example, as illustrated in FIG. 2B and FIG. 2D, the twisted wire structure 160 and the mesh structure 170 are co-disposed within the interthread space 150 and the mesh structure 170 is disposed at the helical thread 140 and the first end 110. As depicted in FIG. 2B, the twisted wire structure 160 is disposed toward the first end 110 and the mesh structure 170 is disposed toward the second end 130 (not shown) within the interthread space 150. As shown in FIG. 2D, the twisted wire structure 160 is disposed toward the second end 130 (not shown) and the mesh structure 170 is disposed toward the first end 110. Further, if desired, both ends of the interthread space 150 could include a mesh structure 170 transitioning to a twisted wire structure 160 in the center region of the interthread space 150. In another example as illustrated in FIG. 20, the twisted wire structure 160 is disposed in the interthread space 150 and the first end 110 and the mesh structure 170 is disposed at the helical thread 140. In another embodiment, the twisted wire structure 160 may be disposed at the second end 130. The arrangement of the twisted wire structure 160 and mesh structure 170 can thus be varied to achieve desired properties of the stent 100.

In certain embodiments, the woven braid 106 can provide for elongation of the stent 100 of less than 150% when the stent 100 is radially crimped from a non-crimped or non-constrained diameter to a crimped or constrained diameter (such as a crimped or constrained diameter of 6.5 French to 8.5 French). For example, the stent 100 having a 100 millimeter length when non-constrained can have a length of less than 150 millimeters when constrained to fit within a 6.5 French to 8.5 French catheter for delivery to a target treatment site, e.g., biliary duct. The elongation of the stent 100 to less than 150% may be facilitated by the combination of sections of the twisted wire structure 160 and sections of the mesh structure 170. The twisted wire structure 160 may have substantially 0% elongation because the wires 114 of the twisted wire pair 161 are oriented with the longitudinal axis 116 of the stent 100 and will not stretch when the stent 100 is crimped. The mesh structure 170 allows the stent 100 to elongate when crimped to a smaller diameter because the cells of the mesh structure 170 elongate. Generally speaking, the higher the ratio of twisted wire structure 160 to the mesh structure 170 along the length of the stent 100 the lower the percent elongation of the stent 100 when constrained.

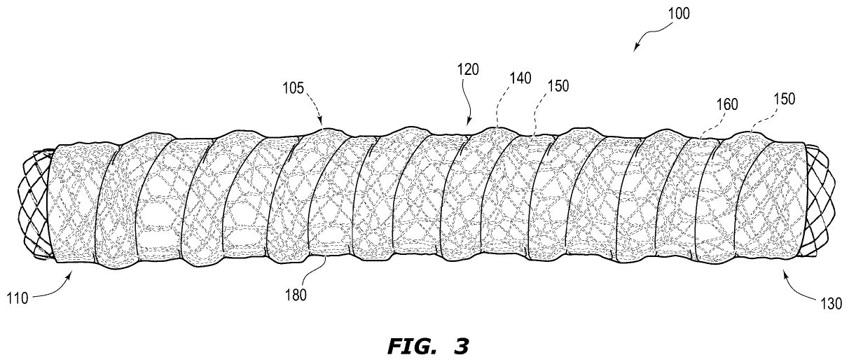

In some embodiments, as illustrated in FIG. 3, the stent 100 may be coated or covered along its entire length or over portions thereof with a cover 180. For example, the cover 180 may cover one or more of the first end 110, the midbody 120, and the second end 130. The cover 180 can comprise a flexible material suitable for placement in a body lumen, such as polyurethane, silicone, polytetrafluoroethylene, or any combination thereof. The cover 180 is coupled to the wires 114 or other material that forms the stent body 105. The cover 180 can further define the interior space of the stent 100 and can facilitate passage of particles or fluid through the lumen of the stent 100.

As illustrated in FIG. 1, the stent 100 can include a loop or handle 190 arranged about the distal or second end 130 of the stent 100. In other embodiments, the handle 190 can be arranged about the proximal or first end 110 and the distal or second end 130. When the stent 100 is deployed into the bile duct, the distal end 130 may be positioned at the opening of the bile duct into the duodenum. The handle 190 can be grasped with forceps to remove the stent 100 from the bile duct or to reposition the stent 100 within the bile duct. As shown in FIG. 1, the handle 190 includes a twisted wire pair 161 coupled to the mesh structure 140. In another embodiment, two, three, or four handles 190 may be arranged about the proximal end 110 and/or distal end 130. For instance, two handles 190 can be arranged about 180 degrees from one another, three handles 190 can be arranged about 120 degrees from one another, or four handles 190 can be arranged about 90 degrees from one another. Other arrangements are also contemplated.

In one embodiment, the helical thread 140 can have a cross-sectional shape, which may be selected with a view to enhancing migration resistance in a particular anatomical structure and/or based on consideration of the materials and method used to fabricate the stent 100. In some embodiments, the helical thread 140 can have a generally circular cross-sectional profile—i.e., the shape of the profile of the helical thread 140 defines at least part of a circle. In some embodiments, the helical thread 140 can have a cross-sectional profile with a generally elliptical geometry. In various embodiments, the helical thread 140 has a generally triangular cross-sectional profile. In some embodiments, the helical thread 140 has a cross-sectional profile with a roughly symmetrical inverted “V” shape. In some embodiments, the helical thread 140 has a buttress-shaped cross-sectional profile.

The cross-sectional profile shapes of the helical thread 140 listed above are not intended to be exhaustive, and it is contemplated that stents in accordance with the present disclosure may include threads having other cross-sectional shapes as well as combinations of the foregoing. With respect to the various cross-sectional profile shapes, the height and breadth of the helical thread 140 in absolute terms may depend to a degree on the overall dimensions of the stent, and the relationship between the height and breadth is determined in part by the profile geometry. For example, in some embodiments the stent 100 comprises an interthread space 150 with a profile based on a circle having a diameter of from about 1 millimeter to about 10 millimeters, such as from about 2 millimeters to about 4 millimeters, or about 3 millimeters. In such embodiments, a maximum height of the helical thread 140 protruding beyond the interthread space 150 is from about 0.20 millimeter to about 1.5 millimeters, such as from about 0.5 millimeter to about 1 millimeter, or about 0.75 millimeter.

The length of the sections of twisted wire structure 160 and the length of the sections of mesh structure 170 can vary to provide desired functional and clinical characteristics. In some embodiments, the length of the mesh structure 170 at one or both ends 110, 130 can range from about two millimeters to about 30 millimeters, or from about five millimeters to about ten millimeters. Along the midbody 120, sections of twisted wire structure 160 ranges from about three millimeters to about seven millimeters, and the length of the sections of mesh structure 170 ranges from about one millimeter to about five millimeters.

FIG. 2E shows the stent 100 having a flange 135 disposed at the second end 130, wherein the flange 135 flares to a diameter substantially equivalent to the diameter of the midbody 120 at the apex of the helical thread 140. The flange 135 can allow a wider opening of the lumen 108 at one or both ends to facilitate fluid flow or access through the stent 100. In another aspect, the flange 135 may contribute to migration resistance by interacting with the surfaces of a lumen in a subject's body. In some embodiments, the flange 135 may be disposed at the first end 110 or at both the first and second ends 110, 130. In certain embodiments, as exemplified by FIG. 2E, the flange 135 has a conical shape that is concentric to the axis 116 of the stent 100. In other embodiments, the flange 135 can have a cylindrical shape. In still other embodiments, the flange 135 can taper back toward the axis 116 of the stent 100.

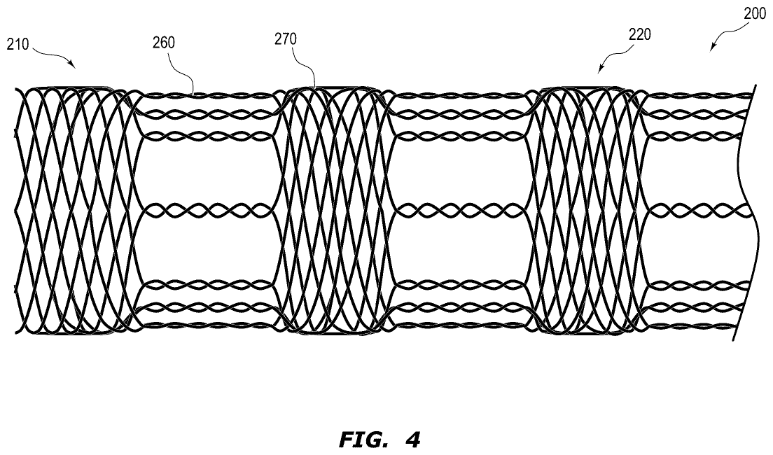

FIG. 4 depicts an embodiment of a stent 200 that resembles the stent 100 described above in certain respects. Accordingly, like features are designated with like reference numerals, with the leading digit incremented to “2.” For example, the embodiment depicted in FIG. 4 includes a woven braid that may, in some respects, resemble the woven braid 106 of FIG. 1. Relevant disclosure set forth above regarding similarly identified features thus may not be repeated hereafter. Moreover, specific features of the stent 100 and related components shown in FIGS. 1-3 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the stent 200 and related components depicted in FIG. 4. Any suitable combination of the features, and variations of the same, described with respect to the stent 100 and related components illustrated in FIGS. 1-3 can be employed with the stent 200 and related components of FIG. 4, and vice versa. This pattern of disclosure applies equally to further embodiments depicted in subsequent figures and described hereafter, wherein the leading digits may be further incremented.

As illustrated in FIG. 4, the stent 200 includes a generally tubular stent body having a midbody 220 disposed between a first end 210 and a second end (not shown). The stent body is formed from a woven braid. The woven braid includes sections of a twisted wire structure 260 and a grid or mesh structure 270 alternatingly distributed along the length of the stent body 205. As illustrated, the outer diameter of the stent body is substantially constant over the length of the stent body. Lengths of the sections of the twisted wire structure 260 and the mesh structure 270 may be equivalent or they may vary over the length of the stent body to provide desired physical characteristics, such as elongation, flexibility, compressibility, etc. The length of the sections of twisted wire structure 260 can be from about three millimeters to about seven millimeters, and the length of the sections of mesh structure 270 can be from about one millimeter to about five millimeters.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “substantially” and “about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration. All ranges also include both endpoints.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A stent comprising:
a tubular body having a longitudinal axis and a midbody extending between a first end and a second end,
wherein a first portion of the midbody comprises a mesh structure, and
wherein a second portion of the midbody comprises a twisted wire structure.

2. The stent of claim 1, wherein the length of the tubular body when radially crimped to 6.5 French diameter to 8.5 French diameter is less than 150% of the length of the tubular body when uncrimped.

3. The stent of claim 1, further comprising a helical thread circumferentially arranged on an outer surface of the midbody, wherein the helical thread comprises a plurality of turns and an interthread space.

4. The stent of claim 3, wherein one or more of the helical thread or the interthread space comprise the mesh structure.

5. The stent of claim 3, wherein one or more of the helical thread or the interthread space comprise the twisted wire structure.

6. The stent of claim 1, wherein one or more of the first end or the second end comprise the mesh structure.

7. The stent of claim 1, wherein one or more of the first end or the second end comprise the twisted wire structure.

8. The stent of claim 1, further comprising a cover surrounding and coupled to one or more of the first end, the second end, or the midbody.

9. The stent of claim 1, wherein one or more of the first end or the second end comprise a stent removal handle extending axially from one or more of the first end or the second end, wherein the stent removal handle comprises the twisted wire structure.

10. The stent of claim 1, wherein one or more of the first end or the second end is flared.

11-12. (canceled)

13. The stent of claim 1, wherein the tubular body further comprises a fluoroscopic marker.

14-15. (canceled)

16. A biliary stent comprising:
a tubular body having a longitudinal axis and a midbody portion extending between a first end portion and a second end portion,
wherein the midbody portion comprises a helical thread circumferentially disposed on at least a section of the midbody portion,
wherein the helical thread comprises a plurality of turns and an interthread space disposed between the plurality of turns,
wherein the helical thread comprises a grid wire structure, and
wherein the interthread space comprises a twisted wire structure.

17. (canceled)

18. The biliary stent of claim 16, wherein one or more of the helical thread or the interthread space comprise the grid wire structure.

19. The biliary stent of claim 16, wherein the helical thread further comprises the twisted wire structure.

20. The biliary stent of claim 16, wherein the interthread space further comprises the grid wire structure.

21. The biliary stent of claim 16, wherein one or more of the first end portion or the second end portion comprise the grid wire structure.

22. The biliary stent of claim 16, wherein one or more of the first end portion or the second end portion comprise the twisted wire structure.

23. (canceled)

24. The biliary stent of claim 16, wherein one or more of the first end portion or the second end portion comprise a stent removal handle extending axially from one or more of the first end portion or the second end portion, wherein the stent removal handle comprises the twisted wire structure.

25. The biliary stent of claim 16, wherein one or more of the first end portion or the second end portion is flared radially outward.

26-30. (canceled)

31. A stent comprising:
a tubular body having a longitudinal axis and a midbody extending between a first end and a second end, the tubular body comprising a helical thread circumferentially arranged on an outer surface of the midbody, wherein the helical thread comprises a plurality of turns and an interthread space;
wherein the helical thread comprises a mesh structure, and
wherein the interthread space comprises a twisted wire structure.
